(19) United States
(12) Patent Application Publication
(10) Pub. No.: US 2015/0290170 A1
(43) Pub. Date: Oct. 15, 2015

(54) KETOROLAC-CONTAINING SUSTAINED RELEASE DRUG DELIVERY SYSTEMS

(71) Applicant: ALLERGAN, INC., Irvine, CA (US)

(72) Inventors: Hui Liu, Irvine, CA (US); Kun Xu, Laguna Niguel, CA (US); Patrick M. Hughes, Aliso Viejo, CA (US)

(73) Assignee: ALLERGAN, INC., Irvine, CA (US)

(21) Appl. No.: 14/438,114

(22) PCT Filed: Oct. 24, 2013
(86) PCT No.: PCT/US2013/066626

§ 371 Date: Apr. 23, 2015

Related U.S. Application Data
(60) Provisional application No. 61/718,811, filed on Oct. 26, 2012.

(51) Int. Cl. A61K 31/407; A61K 47/34; A61K 9/00

(52) U.S. Cl. CPC A61K 31/407; A61K 9/0051; A61K 47/34

(57) ABSTRACT

Biodegradable intraocular drug delivery systems comprising ketorolac and a biodegradable polymer matrix that can release ketorolac into an eye for an extended period of time at a substantially constant rate are described.

### CROSS REFERENCE TO RELATED APPLICATIONS

This application claims the benefit of U.S. Provisional Patent Application Ser. No. 61/718,811, filed on Oct. 26, 2012, the entire disclosure of which is incorporated herein by reference.

### BACKGROUND

The present invention relates to biodegradable intraocular drug delivery systems that provide for the sustained release of ketorolac. The drug delivery systems may be useful for treating chronic diabetic macular edema and to reduce pain and inflammation in an eye following cataract surgery.

Ketorolac is a non-steroidal anti-inflammatory drug that is often prescribed for the treatment of post-operative pain and inflammation. However, effective management of pain and inflammation can require frequent administration of ketorolac. In cases involving ocular surgery, patients may need to administer ketorolac-containing eye drops up to 4 times daily for 2 weeks. Furthermore, as many as four different eye drops may be prescribed by the doctor after an ocular surgery. As a result, patients may not only have difficulty complying with the dosing regimen but may also become confused by the different dosages and administration frequencies.

Nevertheless, ocular inflammation, if not effectively treated, can lead to vision damage. In some instances, macular edema may result. Thus, a biodegradable, biocompatible, and injectable drug delivery system that can deliver a therapeutically effective amount of ketorolac for an extended period after only a single administration would be of great value to patients.

### SUMMARY

Accordingly, the present invention provides for biodegradable intraocular drug delivery systems that provide for the sustained release of ketorolac to the eye for a period of about 3 weeks or more, such as, for example about 1 month, 6 weeks, 2 months, or for about 3 to 6 months.

A drug delivery system according to this invention can comprise or consist of a biodegradable polymer matrix and ketorolac free acid or a pharmaceutically acceptable salt of ketorolac free acid associated with the biodegradable polymer matrix.

For example, in one embodiment the drug delivery system comprises ketorolac tromethamine (formula shown below) associated with a biodegradable polymer matrix.

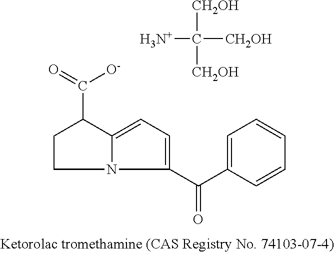 [SMILES: O=C(c1ccccc1)c1ccc2n1CCC2C(=O)[O-] ; [NH3+]C(CO)(CO)CO]

In another embodiment, the drug delivery system comprises ketorolac free acid associated with a biodegradable polymer matrix.

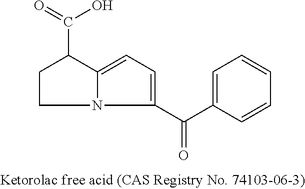 [SMILES: O=C(c1ccccc1)c1ccc2n1CCC2C(=O)O]

The drug delivery system can comprise or consist of a solid or semi-solid intraocular implant configured for placement in an eye of a patient suffering from ocular pain and inflammation associated with cataract surgery, macular edema, chronic diabetic macular edema, or other ocular condition. Examples of solid intraocular implants include extruded implants (sometimes referred to as extruded filaments, fibers or rods) and compressed tablets. An extruded implant can be cylindrical (such as a rod) or non-cylindrical. An extruded intraocular implant can be configured for placement in the vitreous body of the eye. The extruded implant may comprise or consist of ketorolac free acid or ketorolac tromethamine and a biodegradable polymer matrix. Accordingly, the drug delivery system can be an extruded biodegradable implant configured for placement in an eye, such as the vitreous body of an eye. Such an implant may be referred to generally as an intraocular implant and more specifically as an intravitreal implant.

The biodegradable polymer matrix in an implant (and therefore in a drug delivery system) may comprise or consist of one or more biodegradable polymers. For example, the matrix may comprise or consist of first and second biodegradable polymers. The polymers will generally be biocompatible with the eye so as to cause no adverse reactions in the eye. For example, the biodegradable matrix may comprise a poly(D,L-lactide-co-glycolide), a poly(D,L-lactide), or a combination of a poly(D,L-lactide-co-glycolide) and a poly(D,L-lactide). The ketorolac can be homogenously or heterogeneously dispersed and/or dissolved in the biodegradable polymer matrix.

The drug delivery system (for example, an extruded implant) may contain from about 15% to about 50% by weight ketorolac tromethamine or ketorolac free acid and from about 50% to about 85% by weight poly(D,L-lactide-co-glycolide), poly(D,L-lactide), or combination thereof. The drug delivery system is loaded with sufficient ketorolac to release a therapeutically effective amount of ketorolac into the eye of a patient over an extended period of time, which may be for about 3 weeks to about 3 months or more. These systems can promote healing of inflamed tissue following cataract surgery and may further lower the risk of post-surgical complications, including macular edema, after cataract surgery.

In some cases, such as in the treatment of an infection or chronic condition such as macular edema, it may be desirable to provide a relatively constant rate of release of ketorolac from the drug delivery system over the life of the system. For example, it may be desirable for the ketorolac to be released in amounts from about 0.01 μg to about 2 μg, 1 to 30 μg, or about 10 to about 30 μg or more per day for the life of the system (e.g., for about 3 weeks to about 3 months or more).

Accordingly, in one embodiment the invention provides for an extruded biodegradable intraocular implant comprising a biodegradable polymer matrix and ketorolac free acid or ketorolac tromethamine associated with the biodegradable polymer matrix, wherein the implant releases ketorolac at a substantially constant rate, releasing on average about 10 to about 30 μg ketorolac per day with zero order or approximately zero order kinetics (e.g., in a linear fashion), for about 3 weeks.

In another embodiment, the invention provides for an extruded biodegradable intraocular implant comprising a biodegradable polymer matrix and ketorolac free acid or ketorolac tromethamine associated with the biodegradable polymer matrix, wherein the implant releases ketorolac at a substantially constant rate, releasing on average about 1 to about 30 μg ketorolac per day with zero order or approximately zero order kinetics (e.g., in a linear fashion), for about 3 weeks or more.

Extruded implants can weigh about 1 mg or from about 1 to about 2 mg, have a diameter of from about 0.008 inches to about 0.025 inches, a length of about 1 to about 10 mm, and can be suitable for placement in the vitreous body of the eye. In a preferred embodiment an implant is about 0.018″ in diameter, 6 mm in length, and about 1.2 mg in total weight. Because they provide for long term release of ketorolac, these implants may be particularly useful for treating ocular pain and inflammation, macular edema, including chronic diabetic macular edema, without subjecting the patient to more than one administration of the implant.

More generally, the invention provides for an extruded biodegradable intraocular implant for reducing pain and/or inflammation and treating chronic diabetic macular edema in an eye of a mammal. The implant can comprise from about 15% to about 50% by weight ketorolac tromethamine or ketorolac free acid, or a combination of ketorolac tromethamine and ketorolac free acid, and about 50% to about 85% by weight poly(lactide-co-glycolide), polylactide, or a combination thereof. The implant can be configured for placement in the vitreous body of the eye, having a diameter or other smallest dimension (as in the case of non-cylindrical implants) of about 0.008 inches to about 0.030 inches, a length of from about 1 to about 10 mm, and a total weight of from about 0.5 mg to about 2 mg.

A ketorolac drug delivery system described herein may advantageously provide for extended release times of ketorolac, releasing from about 1 μg to about 30 μg, about 1 μg to about 10 μg, or from about 10 μg to about 30 μg of ketorolac per day in a linear fashion (i.e., at a constant or substantially constant rate) for about 3 weeks to about 3 months or more. Useful examples are described in Table 1. Thus, the patient, in whose eye the drug delivery system has been placed, receives a therapeutic amount of ketorolac for an extended time without requiring additional administrations of ketorolac as is typically required with topical formulations. For example, extruded implants of the present invention, upon placement in the vitreous of an eye, can deliver a therapeutically effective amount of ketorolac to the eye for at least about three weeks or longer, such as 1 or 2 months or between 3 and 6 months. The sustained local delivery of the therapeutic agent from the present drug delivery systems reduces the high transient concentrations associated with traditional bolus injection or pulsed dosing.

Ocular conditions that can be treated by the ketorolac-containing drug delivery systems described herein include post-operative ocular pain and inflammation, such as that resulting from cataract surgery, macular edema, diabetic macular edema, chronic diabetic macular edema, infection, and uveitis.

In this regard, one embodiment provides for a method of treating ocular pain and inflammation comprising placing a drug delivery system described herein (such as an extruded biodegradable intraocular implant) into an eye of a mammal (patient) in need thereof. Examples of mammals in need of treatment include those that have received cataract surgery in an eye. In a particular form of this method the drug delivery system is placed in the vitreous body of the eye of the mammal after cataract surgery. The method may have the added benefit of lowering the risk of the mammal developing post-operative macular edema. The implant may not only reduce pain and/or inflammation arising from cataract surgery but also provide for shorter recovery times and more rapid improvement in vision as compared to the administration of ketorolac solely by use of eye drops.

Another embodiment provides for a method of treating macular edema in an eye of a mammal in need thereof, comprising placing a drug delivery system described herein into an eye of the mammal, thereby treating the macular edema and, possibly, improving the visual performance of the eye. The macular edema treated by this method can be a chronic diabetic macular edema. The method can comprise placing a ketorolac-containing implant into the vitreous body of the eye of the mammal afflicted with macular edema.

The invention further provides for a method of making a ketorolac-containing drug delivery system, comprising combining or mixing ketorolac with one, two, or more biodegradable polymers. The mixture may then be extruded or compressed to form a single composition. The single composition may then be processed (e.g. cut to desired lengths or sizes and sterilized) to form individual implants suitable for placement in an eye of a patient.

### BRIEF DESCRIPTION OF THE DRAWINGS

FIG. 1 is a plot of the cumulative release of ketorolac over time for the implant of Example 5-1.

FIG. 2 is a plot of the cumulative release of ketorolac over time for the implant of Example 5-2.

FIG. 3A is a plot of the cumulative release of ketorolac over time for the implant of Example 5-3.

FIG. 3B is a plot of the cumulative release of ketorolac over time for the implant of Example 5-8.

### DETAILED DESCRIPTION

As used herein, “associated with a biodegradable polymer matrix” means mixed with, dissolved and/or dispersed within, encapsulated by, or coupled to.

The term “biodegradable polymer” refers to a polymer or polymers which degrade in vivo, and wherein degradation of the polymer or polymers over time occurs concurrent with or subsequent to release of the therapeutic agent. A biodegradable polymer may be a homopolymer or a copolymer comprising two or more different structural repeating units.

“Suitable or configured for insertion (or implantation) in (or into) an eye, ocular region or site” with regard to an implant, includes an implant which has a size (dimensions) such that it can be inserted or implanted without causing excessive tissue damage and without unduly physically interfering with the existing vision of the patient into which the implant is implanted or inserted.

“Treating” and ‘treatment” includes any beneficial effect in the eye of an individual produced by the present methods, which effect can be a reduction in, retardation of, or relief from one or more signs and/or symptoms of the ocular condition. A reduction of one or more signs and/or symptoms may include, but is not necessarily limited to, a reduction in ocular inflammation, swelling, redness, pain, or discomfort. Treatment may improve the well being, health, visual performance, and/or optical quality of the eye(s) in the individual. The sign(s) and symptom(s) positively affected by the treatment will depend on the particular ocular condition.

An “intraocular implant” refers to a device or element that is configured to be placed in an ocular region of the eye. Examples include extruded filaments, comprising a biodegradable polymer matrix and an active agent, such as ketorolac, associated with the polymer matrix, and cut to a length suitable for placement in an eye. A suitable length for placement in an eye may be from about 1 mm to about 10 mm. Intraocular implants are generally biocompatible with physiological conditions of an eye and do not cause adverse reactions in the eye. In certain forms of the present invention, an intraocular implant may be configured for placement in the vitreous body of the eye. Intraocular implants may be placed in an eye without disrupting vision of the eye. Implants can be biodegradable and may be produced by an extrusion process, as described herein. Implants produced by an extrusion process and comprising ketorolac free acid or ketorolac tromethamine and a biodegradable polymer matrix are examples of a drug delivery system within the scope of the present invention.

The term “biocompatible” means compatible with living tissue or a living system. Biocompatible implants and polymers produce few or no toxic effects, are not injurious, or physiologically reactive and do not cause an immunological reaction.

An “intravitreal” implant is one that is sized, configured, and formulated for placement in the vitreous body of the eye.

The term “pharmaceutically acceptable salts” refers to salts or complexes that retain the desired biological activity of the compound (ketorolac) and exhibit minimal or no undesired toxicological effects to the mammal or cell system to which they are administered. The “pharmaceutically acceptable salts” according to the invention include therapeutically active salt forms of ketorolac. Useful pharmaceutically acceptable salts can include those formed by treating ketorolac free acid with sodium hydroxide, magnesium hydroxide, potassium hydroxide, calcium hydroxide, ammonia and the like; or an organic base such as for example, L-arginine, ethanolamine, betaine, benzathine, morpholine, tromethamine, and the like. Salts formed with zinc are also of potential interest.

As used herein, an “ocular condition” is a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball. An ocular condition can include an inflammation mediated condition.

“Inflammation-mediated” in relation to an ocular condition means any condition of the eye which can benefit from treatment with an anti-inflammatory agent such as ketorolac and is meant to include, but is not limited to, uveitis and macular edema, fungal, bacterial, or viral infections.

The term “therapeutically effective amount” or “effective amount” refers to the level or amount of active agent needed to treat an ocular condition without causing significant negative or adverse side effects to the eye to which the agent is administered.

An “active agent” is a drug substance that produces a therapeutic effect in the patient (human or non-human mammal) to which it is administered.

“Cumulative release profile” means the cumulative total percent of an active agent (such as ketorolac) released from an implant into an ocular region or site in vivo over time or into a specific release medium in vitro over time.

Polylactide, or PLA, includes poly (D-lactide), poly (L-lactide), and poly(D,L-lactide), and may also be identified by CAS Number 26680-10-4.

Poly(lactide-co-glycolide) or PLGA, includes poly(D,L-lactide-co-glycolide), also identified by CAS Number 26780-50-7.

As described above, a drug delivery system according to the present invention comprises or consists of ketorolac free acid or a pharmaceutically acceptable salt thereof associated with a biodegradable polymer matrix. The ketorolac may become associated with the biodegradable polymer matrix by coextrusion with one or more biodegradable polymers to form an extruded implant, as described herein. The drug delivery system can optionally further comprise one or more excipients, such as a preservative, buffering agent, or electrolyte. The drug delivery system (such as an extruded implant) is effective to provide a therapeutically effective amount of ketorolac directly to an ocular region of the eye to treat, prevent, and/or reduce one or more symptoms of an ocular condition for about 3 weeks or 3 months or more. An improvement of the ocular condition obtained by use of drug delivery system described herein may be observed or perceived by a reduction in pain, redness, swelling, or improved visual performance by the patient, or a combination thereof.

A drug delivery system can be in the form of an extruded intraocular implant, an implant produced by an extrusion process cut to a length suitable for placement in an eye of a mammal. The implant can be placed in an eye, such as the vitreous, and can be an extruded cylindrical or non-cylindrical filament (therefore is produced by an extrusion process). The implant may be effective in sustaining release of an amount of ketorolac therapeutically effective for the treatment of the ocular condition for a time period of about three weeks, for about 6 to 8 weeks, or up to 3 months or more after placement in an ocular region. The implants may also be at least somewhat flexible so as to facilitate insertion of the implant into the vitreous of the eye. Intravitreal implants will generally have a diameter in the range of about 0.008 inches (about 0.2 mm) to about 0.030 inches (about 0.75 mm) and a length of about 1 to 10 mm, and weigh from about 500-2000 μg. For example, an intravitreal implant can have a total weight of about 1000-2000 μg. In one embodiment, an intravitreal implant weighs about 1 mg.

Specific examples of polymers that can be used individually or in combination to form the biodegradable polymer matrix of an extruded ketorolac-containing intraocular implant include RESOMER® R203H, 82075, R208, RG502H, and RG7525.

RESOMER® R203H is a poly(D,L-lactide) having acid end groups and an inherent viscosity of about 0.25-0.35 dl/g, as measured for a 0.1% solution in chloroform at 25° C.

RESOMER® R2075 is an ester capped poly(D,L-lactide) having an inherent viscosity of about 1.3-1.7 dl/g, as measured for a 0.1% solution in chloroform at 25° C.

RESOMER® 8208 is an ester capped poly(D,L-lactide) having an inherent viscosity of about 1.8-2.2 dl/g, as measured for a 0.1% solution in chloroform at 25° C.

RESOMER® R502H is a poly(D,L-lactide-co-glycolide) having acid end groups and an inherent viscosity of about 0.16-0.24 dl/g (as measured for a 0.1% solution in chloroform at 25° C.), and a D,L-lactide:glycolide ratio of about 50:50.

RESOMER® RG752S is an ester capped poly(D,L-lactide-co-glycolide) having an inherent viscosity of about 0.16-0.24 dl/g (as measured for a 0.1% solution in chloroform at 25° C.), and a D,L-lactide:glycolide ratio of about 75:25.

In addition to ketorolac, an extruded intraocular implant according to the present invention may optionally include one or more additional therapeutic agents. For example, the systems may include one or more antihistamines, one or more steroids, one or more non-steroidal anti-inflammatory agents, one or more antibiotics, one or more beta blockers, one or more antineoplastic agents, one or more immunosuppressive agents, one or more antiviral agents, one or more antioxidant agents, and mixtures thereof.

Alternatively, the extruded implant may comprise a biodegradable polymer matrix and ketorolac free acid or ketorolac tromethamine as the active agent associated with the biodegradable polymer matrix and comprise no active agent other than ketorolac.

The release kinetics and proportions of active agent, polymer, and any other modifiers in an implant may be empirically determined by formulating several implants with varying proportions. A USP approved method for dissolution or release test can be used to measure the rate of release (USP 23; NF 18 (1995) pp. 1790-1798). For example, using the infinite sink method, a weighed sample of the drug delivery device is added to a measured volume of a solution containing 0.9% NaCl in water (or other appropriate release medium), where the solution volume will be such that the drug concentration after release is less than 20%, and preferably less than 5%, of saturation. The mixture is maintained at 37° C. and stirred slowly to ensure drug release. The appearance of the dissolved drug as a function of time may be followed by various methods known in the art, such as spectrophotometrically, HPLC, mass spectroscopy, etc.

In general, the ketorolac-containing drug delivery systems described herein may be used to treat ocular pain and inflammation, uveitis, macular edema, macular degeneration, retinal detachment, ocular tumors, bacterial, fungal or viral infections, multifocal choroiditis, diabetic retinopathy, proliferative vitreoretinopathy (PVR), sympathetic opthalmia, Vogt Koyanagi-Harada (VKH) syndrome, histoplasmosis, uveal diffusion, and vascular occlusion. For example, the present drug delivery systems can be used to reduce ocular pain and inflammation following cataract surgery. This method may further reduce the risk of developing post-surgical complications arising from cataract surgery such as macular edema and may ensure improved compliance as compared to a therapy of anti-inflammatory eye drops.

One method comprises treating a posterior ocular condition in an eye of a patient in need thereof by (a) inserting a biodegradable implant into a posterior ocular site such as the vitreous body of the eye in the patient with the posterior ocular condition, the biodegradable implant comprising (i) ketorolac free acid or a pharmaceutically acceptable salt thereof, such as ketorolac tromethamine, associated with (ii) a biodegradable polymer or polymers; (b) releasing at least a portion of, or substantially all of the ketorolac from the biodegradable implant; and (c) obtaining an improvement in the posterior ocular condition. The inserting step may be carried out by insertion of the implant through the pars plana and adjacent thereto into the vitreous cavity (or vitreous body). Alternately, the insertion step can be carried out by placing the biodegradable implant in the vitreous body about 2 mm to about 6 mm anterior of the macula and not along the visual axis of incoming light through the pupil. A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Examples of posterior ocular conditions include inflammation, macular edema, cystoid macular edema and diabetic macular edema; multifocal choroiditis; ocular trauma which affects a posterior ocular site or location; ocular tumors and retinal disorders,

Another method for treating persistent macular edema includes (a) inserting a biodegradable intraocular implant into the vitreous of a patient with persistent macular edema, the biodegradable implant comprising (i) ketorolac associated with (ii) a biodegradable PLA polymer, PLGA co-polymer, or a combination thereof; (b) releasing at least a portion of, or substantially all of the ketorolac from the biodegradable implant; and (c) obtaining an improvement in the persistent macular edema. The inserting step may be carried out by insertion of the implant through the pars plana and adjacent thereto in the vitreous cavity. Alternately, the insertion step can be carried out by placing the biodegradable implant into the vitreous about 2 mm to about 6 mm anterior of the macula and not along the visual axis of incoming light through the pupil.

Another method for improving the visual acuity of a patient with persistent macular edema includes (a) inserting a biodegradable implant into the vitreous a patient with persistent macular edema by inserting the biodegradable implant through the pars plana and adjacent thereto in the vitreous cavity or alternately, the insertion step can be carried out by placing the biodegradable implant in the vitreous about 2 mm to about 6 mm anterior of the macula and not along the visual axis of incoming light through the pupil, the biodegradable implant comprising (i) ketorolac associated with (ii) a biodegradable polymer; and (b) releasing ketorolac from the biodegradable implant after the inserting step.

With regard to any of the preceding methods, the mammal (and therefore patient) can be a human or non-human mammal, and the implant used can be any of those set forth in Table 1.

An extruded implant according to the present invention may be inserted into the eye, for example the vitreous of the eye, by a variety of methods, including placement by forceps, by trocar, or syringe. One example of a device that may be used to insert the implants into an eye is disclosed in U.S. Patent Publication No. 2004/0054374. A syringe apparatus including an appropriately sized needle, for example, a 22 gauge needle, a 27 gauge needle or a 30 gauge needle, can be effectively used to inject the drug delivery system into the eye of a human or animal. Repeat injections are often not necessary due to the extended release of ketorolac from the systems. In some embodiments, a hand held applicator is used to insert one or more biodegradable implants into the eye. The hand held applicator typically comprises an 18-30 gauge stainless steel needle, a lever, an actuator, and a plunger. Suitable devices for inserting an implant or implants into a posterior ocular region or site include those disclosed in United States Patent Application Publication No. US 2005-0101967.

Various techniques may be employed to make an intraocular implant. Useful techniques include hot melt extrusion and compression methods. For example, a suitable implant can be made using the methods and materials set forth in U.S. patent application publication number 2004/0137059. Extrusion methods may use temperatures of about 25° C. to about 150° C., or about 60° C. to about 130° C. The extruded filament or rod is then cut to a desired length suitable for placement in the eye.

Some biodegradable implants may be capable of releasing a therapeutically effective amount of ketorolac, such as about 1-30 μg, 1-10 μg, or 10-30 μg of ketorolac tromethamine or ketorolac free acid per day, at a constant rate, for about 3 weeks or about 3 months or more. The duration of release for these implants may vary depending upon factors such as the size of the implant and the amount of the ketorolac in the implant. The release rate may be constant over this period resulting in a linear cumulative release rate profile.

The preparation of ketorolac is described in U.S. Pat. No. 6,197,976. Biodegradable polymers from the RESOMER® product line used for the implant can be obtained from Evonik Industries AG, Germany, for example.

The present invention includes, but is not limited to, the following embodiments (1-13):

1. An extruded biodegradable intraocular implant comprising ketorolac free acid or ketorolac tromethamine associated with a biodegradable polymer matrix, wherein the implant releases about 1 to about 30 μg of ketorolac per day for at least 2 months after placement in an eye of a mammal.

2. An implant according to embodiment 1, wherein the implant comprises about 45% by weight ketorolac free acid and about 55% by weight of an ester capped poly(D,L-lactide) having an inherent viscosity of about 1.8-2.2 dl/g (RESOMER® R208).

3. An extruded biodegradable intraocular implant comprising ketorolac free acid or ketorolac tromethamine associated with a biodegradable polymer matrix, wherein the implant releases about 10 to about 30 μg of ketorolac per day for at least 3 weeks after placement in the eye of a mammal.

4. An implant according to embodiment 3, wherein the implant comprises about 30% by weight ketorolac tromethamine and about 70% by weight of an ester capped poly(D,L-lactide-co-glycolide) having an inherent viscosity of about 0.16-0.24 dl/g and a D,L-lactide:glycolide ratio of about 75:25 (RESOMER® RG752S).

5. An implant according to any of embodiments 1-4, wherein the implant releases ketorolac at a substantially constant rate for about 3 weeks after placement of the implant in an eye of a mammal.

6. An implant according to any of embodiments 1-4, wherein the implant releases ketorolac at a substantially constant rate for about 1 month after placement of the implant in an eye of a mammal.

7. An implant according to any of embodiments 1-6, wherein the implant has a total weight of about 0.5 mg to about 2 mg.

8. An implant according to any of embodiments 1-7, wherein the implant is effective for reducing pain and inflammation in the eye after cataract surgery on the eye for 3 weeks or more after placement of the system in the eye.

9. A method for treating pain, inflammation, or macular edema in an eye of a mammal, comprising inserting into the vitreous body of the eye of the mammal an implant according to any of embodiments 1-8, thereby reducing the pain or inflammation, or treating the macular edema in the eye.

10. The method of embodiment 9, wherein the implant delivers from about 1 μg to about 30 μg ketorolac per day for about 3 weeks, 1 month, 2 months, or 3 months or more after placement of the system in the eye.

11. A method according to embodiment 9, wherein the implant delivers from about 10 μg to about 30 μg ketorolac per day for about 3 weeks or more after placement of the system in the eye.

12. A method according to any of embodiments 9-11, wherein the implant is inserted into the vitreous body of an eye following cataract surgery on the eye and wherein the implant is effective for reducing pain and inflammation in the eye for about 3 weeks or more.

13. A method according to any of embodiments 9-12, wherein the mammal is a human.

### EXAMPLE 1

### Manufacture and In Vitro Release Measurements of Ketorolac-Containing Implants

Ketorolac-containing implants were made by hot-melt extrusion using a mechanically driven ram micro extruder or a twin drive extruder. The implants were rod shaped, but they can be made into any geometric shape by changing the extrusion or compression die. Polymers were used as received from Boehringer Ingelheim (Resomer®). The drug and polymer were thoroughly mixed in a small container with a spatula. The mixture was then transferred into a stainless steel container containing two ¼ inch stainless steel balls and mixing continued using a Turbula mixer for two separate 15 minute cycles. The powder blend was manually mixed again with a spatula after the first and the final cycle. The blended material was then compacted into an extruder barrel using a special powder compactor. Then the extruder barrel was placed into the heated well (between 60 and 105° C.) of the piston extruder and extruded using a 420 μm nozzle and a speed setting number of 0.0025. Smaller implants were manufactured by using a smaller nozzle or pulling at a faster rate. The diameters of implants range from 0.008 inches to 0.025 inches. They can be cut into pieces at different lengths. Examples of implants produced according to this extrusion process are listed in Table 1. In one example, ketorolac tromethamine (0.3 g) and Resomer RG752S (0.7 g) were well mixed and extruded through a piston extruder at 65° C. The percentage of ketorolac by weight (% w/w) in each of the implants in Table 1 is based on the total weight of the drug substance (ketorolac free acid or ketorolac tromethamine).

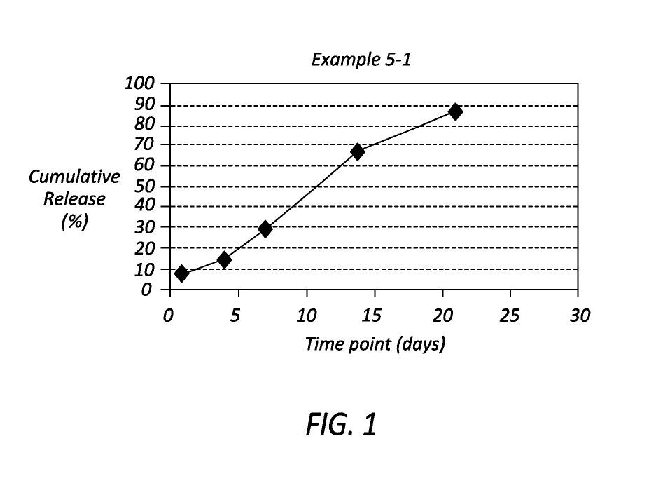

The in vitro release of ketorolac from the implants was measured from triplicates of about 1 mg rods cut from extruded filaments. The rod was placed into a 10 mL vial containing 0.01 M phosphate buffered saline (pH 7.4). The samples were then transferred into a shaking water bath set at 37° C. and 50 rpm. At various time-points, the solution was removed and analyzed by HPLC for the amount of released Ketorolac. The removed solution was replaced with fresh phosphate buffered saline solution. The cumulative release profiles for implants having the formulations of Examples 5-1, 5-2, 5-3, and 5-8 are shown in FIGS. 1-3.

FIG. 1 is a plot of the cumulative release of ketorolac in vitro over time for the implant of Example 5-1.

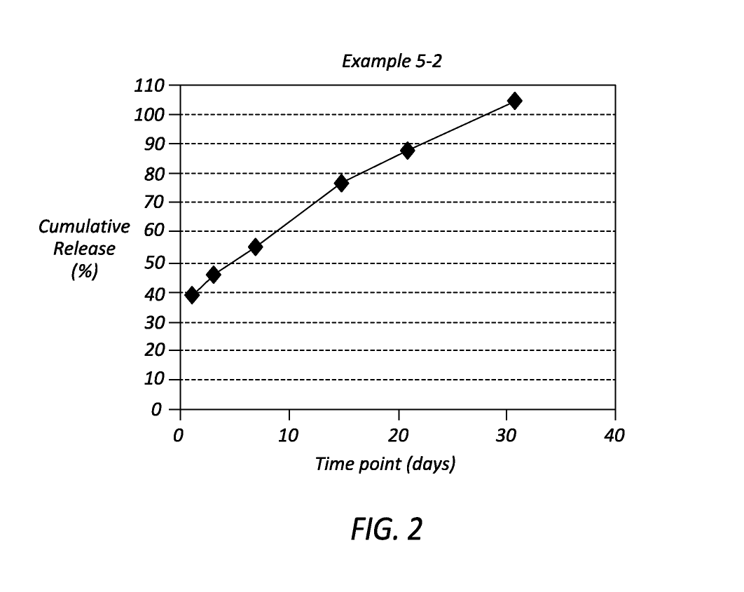

FIG. 2 is a plot of the cumulative release of ketorolac in vitro over time for the implant of Example 5-2.

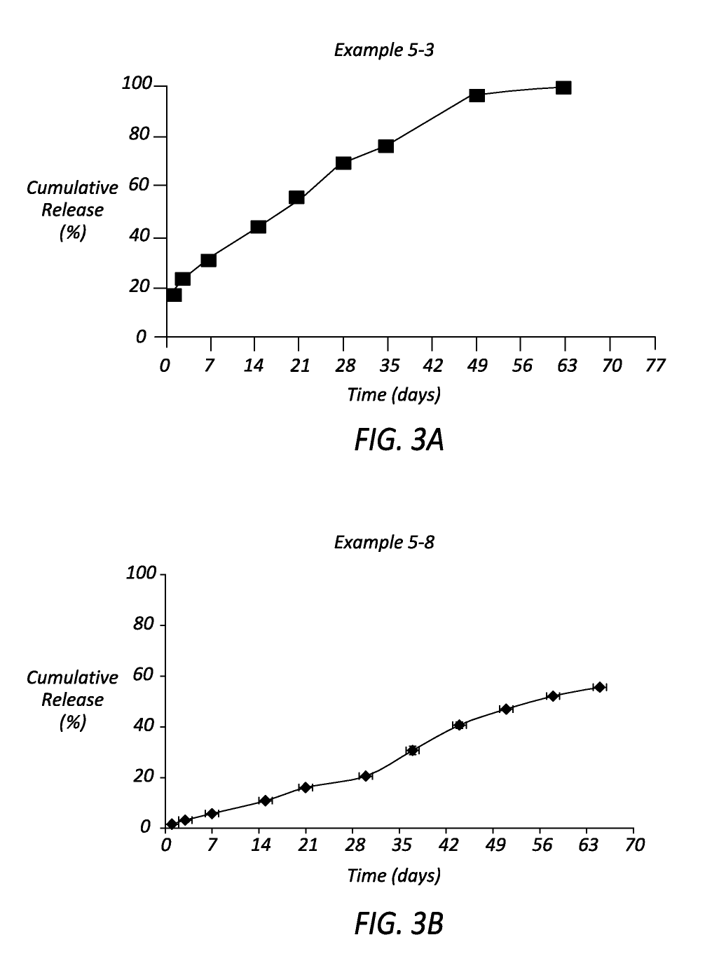

FIG. 3A is a plot of the cumulative release of ketorolac in vitro over time for the implant of Example 5-3.

FIG. 3B is a plot of the cumulative release of ketorolac in vitro over time for the implant of Example 5-8.

TABLE 1
Ketorolac Implants

| | Composition (% w/w) | | | |
|---|---|---|---|---|
| | Ketorolac | | Release | |
| Example | (amount and form) | Polymer composition | duration | |
| 5-1 | 30% tromethamine salt | 70% PLGA (RG752S) | 3 | weeks |
| 5-2 | 50% tromethamine salt | 50% PLGA (RG752S) | 1 | month |
| 5-3 | 40% free acid | 55% PLA (R203H) | 8 | weeks |
| | | 5% PLGA (RG502H) | | |
| 5-4 | 30% free acid | 35% PLA (R203H) | 1 | month |
| | | 35% PLGA (RG752S) | | |
| 5-5 | 45% free acid | 50% PLA (R203H) | 2 | months |
| | | 5% PLGA (RG502H) | | |
| 5-6 | 40% free acid | 52% PLA (R203H) | 6 | weeks |
| | | 8% PLGA (RG502H) | | |
| 5-7 | 40% free acid | 58% PLA (R207S) | 3-6 | months |
| | | 2% PLGA (RG502H) | | |
| 5-8 | 45% free acid | 55% PLA (R208) | 3-6 | months |

1. An extruded biodegradable intraocular implant comprising;
(a) ketorolac free acid or ketorolac tromethamine; and
(b) a biodegradable polymer matrix;
wherein the implant releases about 1 to about 30 μg of ketorolac per day for at least 2 months after placing the implant in an eye of a mammal.

2. The implant according to claim 1, wherein the implant comprises about 45% by weight ketorolac free acid and about 55% by weight of an ester capped poly(D,L-lactide) polymer having an inherent viscosity of about 1.8 to about 2.2 dl/g.

3. An extruded biodegradable intraocular implant comprising:
(a) ketorolac free acid or ketorolac tromethamine; and
(b) a biodegradable polymer matrix;
wherein the implant releases about 10 to about 30 μg of ketorolac per day for at least 3 weeks after placing the implant in an eye of a mammal.

4. The implant according to claim 3, wherein the implant comprises about 30% by weight ketorolac tromethamine and about 70% by weight of an ester capped poly(D,L-lactide-co-glycolide) polymer having an inherent viscosity of about 1.6 to about 0.24 dl/g and a D,L-lactide:glycolide ratio of about 75:25.

5. The implant according to claim 1, wherein the implant releases ketorolac at a substantially constant rate for about 2 months after the implant in the eye of the mammal.

6. The implant according to claim 3, wherein the implant releases ketorolac at a substantially constant rate for about 3 weeks after placing the implant in the eye of the mammal.

7. The implant according to claim 5, wherein the implant has a total weight of about 0.5 mg to about 2 mg.

8. The implant according to claim 7, wherein the implant is effective for reducing pain and inflammation in the eye after cataract surgery on the eye for about 3 weeks after placing the implant in the eye of the mammal.

9. A method for treating pain, inflammation, or macular edema in an eye of a mammal, comprising inserting an implant according to claim 5 into the vitreous body of the eye of the mammal, thereby reducing pain or inflammation, or treating the macular edema in the eye of the mammal.

10. The method according to claim 9, wherein the implant delivers from about 1 mg to about 30 mg ketorolac per day for about 1 month, about 2 months, or about 3 months after placing the implant in the eye.

11. The method according to claim 10, wherein the implant delivers from about 10 mg to about 30 mg ketorolac per day for about 3 weeks after placing the implant in the eye.

12. The method according to claim 10, wherein the implant is inserted into the vitreous body of the eye following cataract surgery on the eye, and wherein the implant is effective for reducing pain and inflammation in the eye for about 3 weeks.

13. The implant according to claim 6, wherein the implant has a total weight of about 0.5 mg to about 2 mg.

14. The implant according to claim 13, wherein the implant is effective for reducing pain and inflammation in the eye after cataract surgery on the eye for about 3 weeks after placing the implant in the eye of the mammal.

15. A method for treating pain, inflammation, or macular edema in an eye of a mammal, comprising inserting an implant according to claim 6 into the vitreous body of the eye of the mammal, thereby reducing pain or inflammation, or treating the macular edema in the eye of the mammal.

16. The method according to claim 15, wherein the implant delivers from about 1 mg to about 30 mg ketorolac per day for about 1 month, about 2 months, or about 3 months after placing the implant in the eye.

17. The method according to claim 16, wherein the implant delivers from about 10 mg to about 30 mg ketorolac per day for about 3 weeks after placing the implant in the eye.

18. The method according to claim 16, wherein the implant is inserted into the vitreous body of the eye following cataract surgery on the eye, and wherein the implant is effective for reducing pain and inflammation in the eye for about 3 weeks.
